(19) United States
(12) Patent Application Publication
(10) Pub. No.: US 2014/0243970 A1
(43) Pub. Date: Aug. 28, 2014

(54) PROGRAMMING OF BACKUP CONTROL UNIT FOR CARDIAC ASSIST SYSTEM

(71) Applicant: THORATEC CORPORATION, Pleasanton, CA (US)

(72) Inventor: Masamichi Yanai, Ann Arbor, MI (US)

(73) Assignee: THORATEC CORPORATION, Pleasanton, CA (US)

(21) Appl. No.: 13/775,622

(22) Filed: Feb. 25, 2013

(51) Int. Cl. A61M 1/10

(52) U.S. Cl. CPC A61M 1/1086
USPC 623/3.28

(57) ABSTRACT

Upon failure of a primary control unit for controlling an implanted pump in a ventricle assist system enhancing blood flow in a patient, an initial backup control unit is substituted for the failed unit. An interface device is connected to one of the initial backup control unit or the primary control unit. Patient-unique data is downloaded to the interface device to create a transfer data set. The initial backup control unit is connected to the implanted pump as a new primary control unit. The interface device is connected to a replacement backup control unit. The transfer data set is uploaded from the interface device to the replacement backup control unit. The transfer data set on the interface device is modified after uploading to prevent a subsequent uploading from the interface device to another control unit.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to circulatory assist systems, and, more specifically, to configuring a backup control unit when a primary control unit experiences a fault or is otherwise replaced.

Many types of circulatory assist systems are available for either short term or long term support for patients having cardiovascular disease. For example, a heart pump system known as a left ventricular assist device (LVAD) can provide long term patient support with an implantable pump associated with an externally-worn pump control unit and batteries. The LVAD improves circulation throughout the body by assisting the left side of the heart in pumping blood. One such system is the DuraHeart® LVAS system made by Terumo Heart, Inc., of Ann Arbor, Mich. The DuraHeart® system employs a centrifugal pump with a magnetically levitated impeller to pump blood from the left ventricle to the aorta. An electric motor magnetically coupled to the impeller is driven at a speed appropriate to obtain the desired blood flow through the pump.

A typical cardiac assist system includes a pumping unit, electrical motor (e.g., a brushless DC motor integrated in the pump housing), drive electronics, microprocessor control unit, and an energy source such as rechargeable batteries and/or an AC power conditioning circuit. A portion of the system is implanted during a surgical procedure in which a centrifugal pump is placed in the patient's chest. An inflow conduit is pierced into the left ventricle to supply blood to the pump. One end of an outflow conduit is mechanically fitted to the pump outlet and the other end is surgically attached to the patient's aorta by anastomosis. A percutaneous cable connects to the pump/motor unit, exits the patient through an incision, and connects to the external control unit.

A control system for varying pump speed to achieve a target blood flow based on physiologic conditions is shown in U.S. Pat. No. 7,160,243, issued Jan. 9, 2007, which is incorporated herein by reference in its entirety. A target blood flow rate may be established based on the patient's heart rate so that the physiologic demand is met. The control unit may establish a speed setpoint for the pump motor to achieve the target flow. It is essential to automatically monitor pump performance to ensure that life support functions are maintained.

Each external control unit is customizable according to the physiologic needs of the patient based on patient-unique data that is electronically stored in the control unit. Such parameters may include a nominal pump speed setting, upper and lower speed limits, parameters for calculating pump speed according to physiologic variables of the patient, flow threshold for triggering low flow alarm, and patient information such as name, ID number, hospital name, date of birth, height, weight, surgical data, and more. Unique data may also be stored relating to the implant such as pump flow estimation coefficients and a pump part number and/or serial number for device traceability. The stored data may be used during normal pump operation or may be accessed by other devices such as a hospital console that can be connected to the control unit.

Because of the safety critical nature of the external control unit, a dedicated backup control unit is often configured and kept with or near the patient so that in the event of the occurrence of any serious fault in the primary control unit it can be swapped out with the backup. All the same patient-unique data is pre-stored in the backup control unit so that it can be connected to the implanted portion of the system as soon as the faulted primary control unit is removed. Typically, the backup control unit may be programmed with the required data at the same time as the primary unit, i.e., at the time of surgical implantation at the hospital using the same hardware devices.

In the event that a primary control unit fails and is swapped for an initial backup control unit, the need arises for securing and programming a replacement backup control unit to be used in the event of a fault occurrence with the active unit. The same equipment that programmed the initial backup unit may or may not be readily available for configuring the replacement backup. Manual configuration of the patient-unique data onto a replacement unit would be time consuming and subject to manual errors. The same or a different hospital console unit could read the patient-unique data from one of the original control units and transfer it in the replacement backup unit, or a handheld memory transfer module can be used. However, the presence on such a programming unit of multiple sets of data transmitted to or from different control units raises concerns over the possibility of configuring a backup control unit with an incorrect data set.

### SUMMARY OF THE INVENTION

In one aspect of the invention, a method is provided for substituting an initial backup control unit for a primary control unit for controlling an implanted pump in a ventricle assist system enhancing blood flow in a patient. In response to a failure of the primary control unit, the initial backup control unit is connected to the implanted pump as a new primary control unit. An interface device is connected to one of the initial backup control unit or the faulted primary control unit. Patient-unique data is downloaded to the interface device to create a transfer data set. The interface device is connected to a replacement backup control unit. The transfer data set is uploaded from the interface device to the replacement backup control unit. The transfer data set on the interface device is modified after uploading to prevent a subsequent uploading from the interface device to another control unit.

### BRIEF DESCRIPTION OF THE DRAWINGS

FIG. 1 is a diagram of a circulatory assist system with a primary control unit and a backup control unit being configured at a time of surgical implantation.

FIG. 2 is a block diagram showing a portion of a control unit in greater detail.

FIG. 3 illustrates the swapping of a backup unit for a faulted primary unit and the subsequent programming of a replacement backup unit.

FIG. 4 is a block diagram showing an interface device for programming a backup control unit.

FIG. 5 is a flowchart showing one preferred method of the invention.

FIG. 6 is a flowchart showing an alternative method.

### DETAILED DESCRIPTION OF PREFERRED EMBODIMENTS

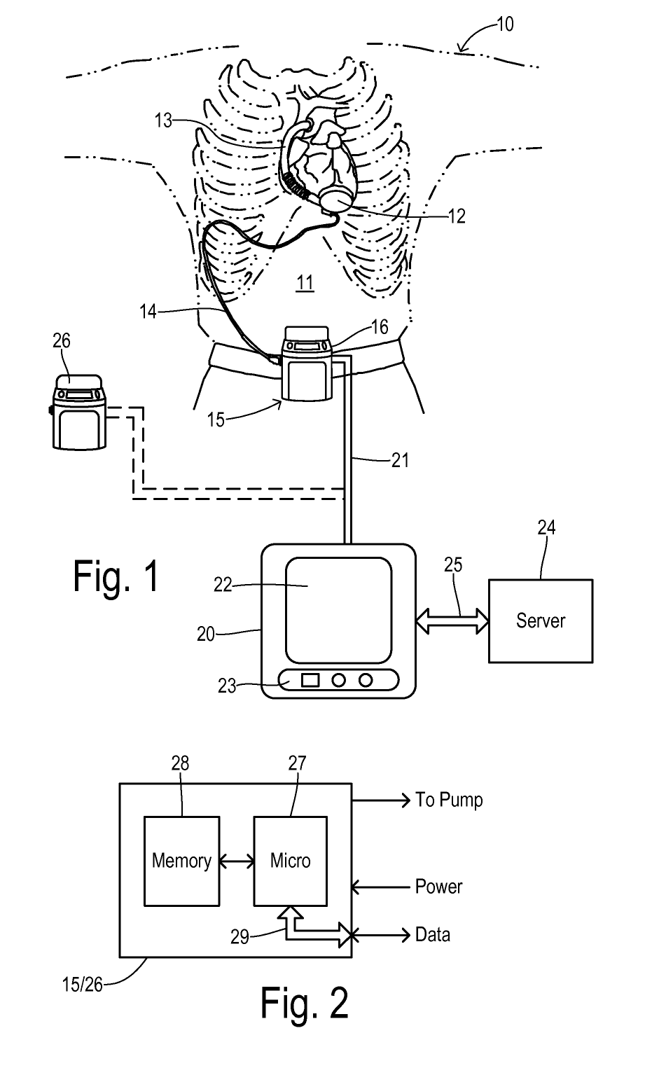

Referring to FIG. 1, a patient 10 is shown in fragmentary front elevational view. Surgically implanted either into the patient's abdominal cavity or pericardium 11 is a pumping unit 12 of a ventricular assist device. An inflow conduit (on the hidden side of unit 12) pierces the heart to convey blood from the patient's left ventricle into pumping unit 12. An outflow conduit 13 conveys blood from pumping unit 12 to the patient's aorta. A percutaneous power cable 14 extends from pumping unit 12 outwardly of the patient's body via an incision to a compact control unit 15 worn by patient 10. Control unit 15 is powered by a main battery pack 16 and/or an external AC power supply and an internal backup battery. Control unit 15 includes a commutator circuit for driving a motor within pumping unit 12. A current sensor is associated with the commutator circuit in order to provide the current measurements on which estimates of the blood flow rate are based.

Control unit 15 further includes internal memory that must be configured with patient-unique and system-unique data to be used for system operation and for providing access for reading the data during subsequent patient care. At or just prior to the time of implantation, controller 16 is connected to a hospital console 20 via a connecting cable 21 for the purpose of programming the data. The data may be manually entered using a human machine interface (HMI) in hospital console 20 (e.g., a touchscreen 22 and manual control elements 23) and/or obtained from a patient database in a separate server 24 connected via a local area network 25.

A substantially identical backup control unit 26 is also connected to hospital console 20 via cable 21 in order to program it with the same patient-unique data and pump-unique data for the implanted pump. Subsequently, backup control unit 26 is kept at the ready in order to substitute it for control unit 15 in the event of an emergency or routine replacement.

FIG. 2 shows that control units 15 or 26 preferably include a microcontroller 27 coupled to a memory 28 which stores the patient-unique and pump-unique data. The control unit has a cable connection to the implantable pump, has a terminal for receiving external power, and has a data terminal connecting with a data bus 29 for external input/output of the contents of memory 28.

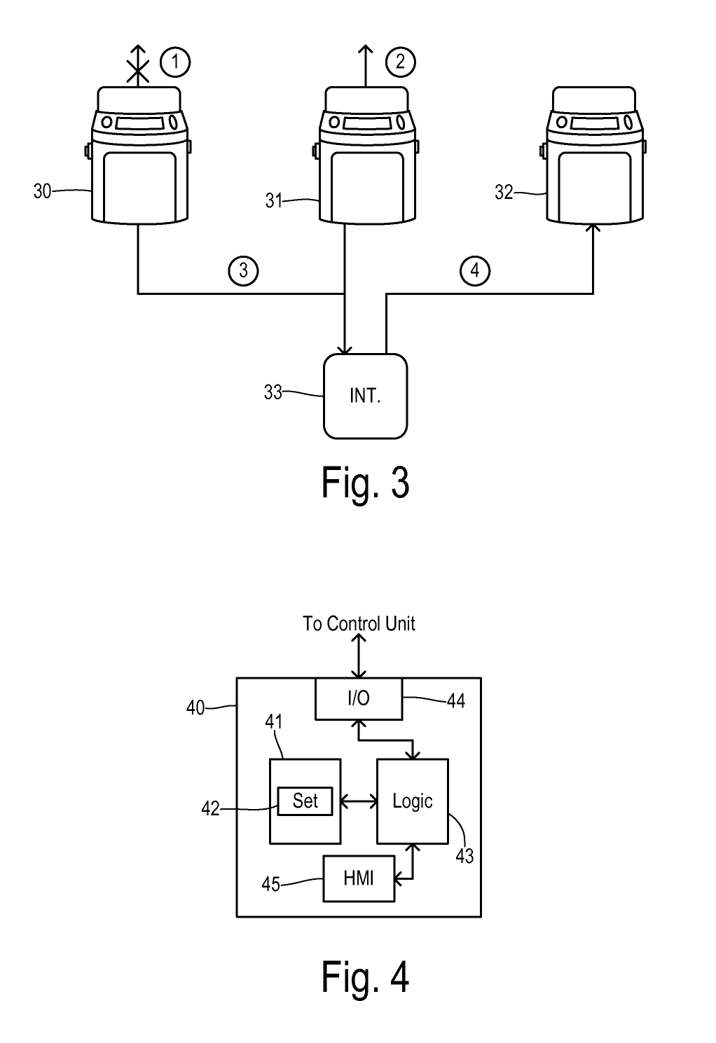

In the event that a fault occurs in a primary control unit, it must be replaced with the backup control unit and then a new backup control unit must be obtained and configured. Various protocols can be established for replacing a faulted controller. One preferred scenario proceeds as shown in FIG. 3. An initial primary control unit 30 generates an alarm in response to self-diagnostic routines to alert a user to the existence of a fault. The alarm conveys the message that control unit 30 should be replaced. In a first step •, the percutaneous connection of control unit 30 with the implanted pump is disconnected, along with any other power or data connections in place. In a second step •, an initial backup control unit 31 is connected to the percutaneous cable of the implanted pump so that operation of the cardiac assist system is restored.

Since the patient has had to swap their backup unit for the primary unit, they may no longer have a backup unit readily available. At a first reasonable opportunity, they should restore their backup protection by obtaining a replacement backup control unit 32. Typically, a visit to the hospital or other care center would be required in order to secure the replacement backup and to have it programmed for the patient.

In a third step •, an interface device 33 at the hospital or other care center is connected to one of control unit 30 or control unit 31 in order to propagate the patient-unique and pump-unique data to the new backup. Interface device 33 may be any electronic unit capable of reading and writing data over a cable connection. It may preferably be comprised of the hospital console specifically designed to work together with the control unit. Device 33 could alternatively be a portable, handheld memory unit designed to only perform the function of duplicating the patient and pump data. Upon connection with control unit 30 or 31, interface device 33 downloads the patient-unique/pump-unique data and stores it as a transfer data set. In a fourth step •, the downloaded data set from one of the initial control units is uploaded from interface device 33 to replacement backup control unit 32.

FIG. 4 shows an interface device 40 in greater detail including a memory block 41 that may store one or more transfer data sets 42. A logic controller 43 is connected to memory 41 and to an input/output terminal interface 44 that can be selectably connected to various control units. An HMI 45 is connected to logic controller 43 to allow for the manual control of data transfers (and for optional data entry for confirming the validity of data sets as explained below). When device 40 is part of a hospital console, then it may contain many other elements and functions as known in the art.

Since interface device 40 is connectable to multiple control units and could potentially have memory contents corresponding to patient and pump data from multiple patients, a potential problem arises in which incorrect data could be copied to a control unit. The present invention provides certain precautions to avoid this potential problem.

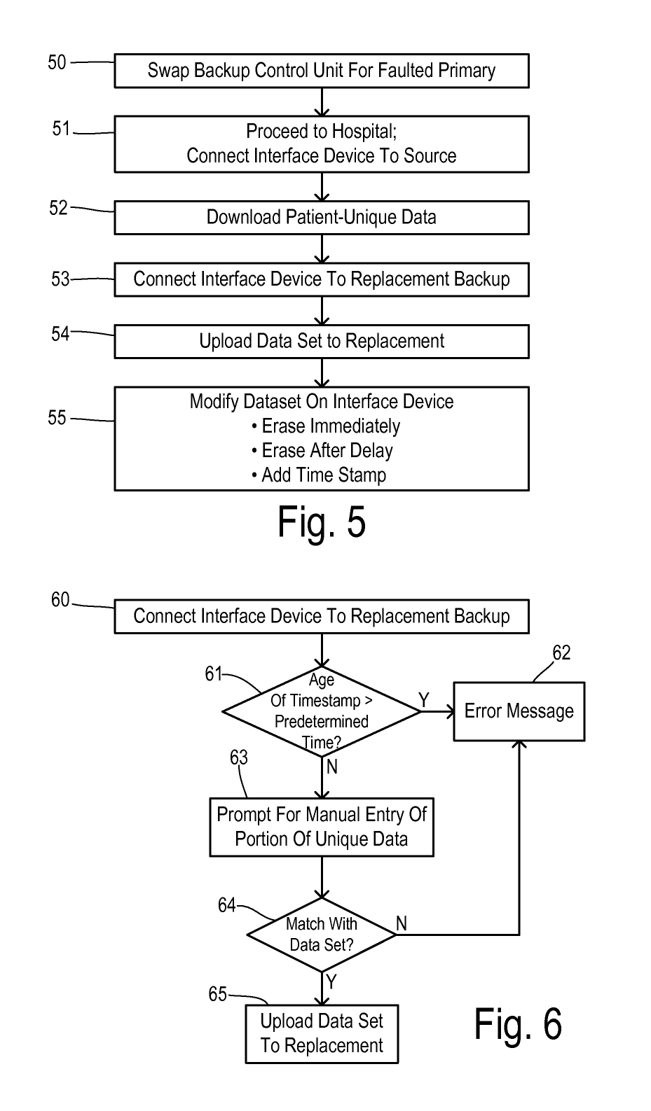

FIG. 5 shows a first embodiment of a method of the invention. After the occurrence of a fault or a warning message generated by a primary control unit, a patient or their helper swaps the faulted control unit for their backup control unit in step 50. Normal system operation is restored using the backup unit. In step 51, the patient would then proceed to a hospital or other care center where a replacement backup unit can be obtained (usually taking the faulted control unit with them), and at the hospital the interface device is connected to either the faulted primary control unit or backup control unit as a source of the patient-unique and pump-unique data. In step 52, the unique data is downloaded to the interface device as a new transfer data set.

The interface device is connected to a replacement backup control unit in step 53. In step 54, the interface device uploads the transfer data set to the replacement control unit, and the replacement unit is held by the patient for possible future use in the event of a fault occurrence in the currently-used control unit (which was formerly the backup unit). Following the upload, the transfer data set on the interface device is modified in step 55 to prevent a subsequent uploading from the interface device to another control unit. In one embodiment, the transfer data set may be erased from the interface device immediately upon a successful upload to the replacement unit. As used herein, erase means to either overwrite the data or make it inaccessible by deleting an entry from a file allocation table, for example. In another embodiment, erasing the transfer data set may occur after a predetermined delay (e.g., 15 minutes) following a successful upload, which would allow a brief interval in which the transfer data set could be propagated to additional devices such as another replacement control unit.

In yet another embodiment, the modification of the transfer data set may be comprised of the appending of a time stamp to the transfer data set. The time stamp preferably indicates a date and time-of-day when the patient and pump data was downloaded from the faulted primary or initial backup control unit. In the event that a time stamp is used, step 54 of FIG. 5 would be modified to perform a test based on the time stamp as shown in FIG. 6. FIG. 6 also shows an optional enhancement for ensuring that an uploaded transfer data set corresponds to the desired patient using a manual verification. In step 60, the interface device is connected to the replacement backup control unit. Before uploading anything, a check is performed in step 61 to determine whether the age of the time stamp appended to the transfer data set is greater than a predetermined age. If so, then an error message is generated at step 62 and the data transfer process ends. If the time stamp is sufficiently new, then an optional manual confirmation of the identity of the patient begins in step 63 wherein the interface device prompts a user for manual entry of a portion of the unique data such as the patient's name or ID number. In response to the prompt, a user enters a manual key sequence according to the desired portion of the unique data (e.g., the patient's name). In step 64, the entered manual key sequence is compared with the selected portion of the transfer data set. If they do not match, then an error message is generated in step 62. If a match is found then the transfer data set is uploaded to the replacement control unit in step 65. As a result, duplication of the correct patient-unique and pump-unique data onto the replacement backup control unit is assured.

What is claimed is:

1. A method of programming a control unit of a ventricle assist system having a pump implanted in a patient, wherein after a fault occurs in a primary control unit then an initial backup control unit is connected to the implanted pump as a new primary control unit, the method comprising the steps of:
connecting an interface device to one of the initial backup control unit or the faulted primary control unit;
downloading patient-unique data to the interface device to create a transfer data set;
connecting the interface device to a replacement backup control unit;
uploading the transfer data set from the interface device to the replacement backup control unit;
modifying the transfer data set on the interface device after uploading to prevent a subsequent uploading from the interface device to another control unit.

2. The method of claim 1 wherein the modifying step is comprised of erasing the transfer data set.

3. The method of claim 2 wherein erasing the transfer data set occurs immediately after the uploading step.

4. The method of claim 2 wherein erasing the transfer data set occurs following a predetermined delay after the uploading step.

5. The method of claim 1 wherein the modifying step is comprised of adding a time stamp to the transfer data set.

6. The method of claim 5 wherein the uploading step further comprises checking the transfer data set for a time stamp and wherein uploading to the replacement backup control unit is performed only if the transfer data set does not include a time stamp having an age greater than a predetermined time.

7. The method of claim 1 further comprising the steps of:
a user entering a manual key sequence on the interface device corresponding to a selected portion of the transfer data set; and
comparing the entered manual key sequence with the selected portion of the transfer data set;
wherein the uploading step is performed only if the comparing step detects a match.

8. An interface device for replacing a backup control unit that has been substituted for a primary control unit for controlling an implanted pump in a ventricle assist system for a patient, comprising:
a transfer terminal for selectably connecting to the primary control unit or backup control units;
a memory for storing a transfer data set; and
a logic controller for 1) initiating a download of the patient-unique data to form the transfer data set from an initial connection with an initial backup control unit or the primary control unit, 2) uploading the transfer data set from the interface device to a replacement backup control unit, and 3) modifying the transfer data set after uploading to prevent a subsequent uploading from the interface device to another control unit, so that the primary control unit can be replaced with one of the backup control units in the event the primary control unit fails.

9. The interface device of claim 8 wherein the logic controller modifies the transfer data set by erasing.

10. The interface device of claim 9 wherein erasing the transfer data set occurs immediately after uploading to the replacement backup control unit.

11. The interface device of claim 9 wherein erasing the transfer data set occurs following a predetermined delay after uploading to the replacement backup control unit.

12. The interface device of claim 8 wherein the logic controller modifies the transfer data set by adding a time stamp.

13. The interface device of claim 12 wherein the logic controller checks a transfer data set for a time stamp prior to uploading to the replacement backup control unit, and the uploading is performed only if the transfer data set does not include a time stamp having an age greater than a predetermined time.

14. The interface device of claim 8 further comprising:
a user interface for entering a manual key sequence;
wherein the logic controller compares the entered manual key sequence with a selected portion of the transfer data set, and wherein the uploading is performed only if the comparison detects a match.

15. The interface device of claim 8 incorporated into a hospital control console.

16. The interface device of claim 8 incorporated into a handheld module.
